(19) United States
(12) Patent Application Publication
(10) Pub. No.: US 2023/0372675 A1
(43) Pub. Date: Nov. 23, 2023

(54) Vascular Access System With Integrated Catheter, Extension Set Providing Integrated Catheter Septum Access, Stabilization Features and Direct Probe Access

(71) Applicant: Becton, Dickinson and Company, Franklin Lakes, NJ (US)

(72) Inventors: Jonathan Karl Burkholz, Salt Lake City, UT (US); Erica E. Neumann, Sandy, UT (US); Curtis H. Blanchard, Herriman, UT (US); Sakthivel Karthikeyan, Bangalore (IN); Deepan Vikash, Nagercoil (IN)

(73) Assignee: Becton, Dickinson and Company, Franklin Lakes, NJ (US)

(21) Appl. No.: 18/198,435

(22) Filed: May 17, 2023

(30) Foreign Application Priority Data
May 18, 2022 (IN) 202211028612

(51) Int. Cl. A61M 25/02; A61B 5/15; A61M 39/02; A61M 25/06

(52) U.S. Cl. CPC A61M 25/02; A61B 5/150992; A61M 39/0247; A61M 25/0606; A61B 5/150007; A61M 2039/0258; A61M 2039/0261

(57) ABSTRACT

A vascular access system for use with a blood draw or probe delivery device, the system including a catheter adapter having a proximal end portion and a distal end portion, wherein a catheter extends from the distal end portion of the catheter adapter, and an extension set. The extension set includes a stabilization platform, a catheter adapter interface, wherein the catheter adapter interface is configured to secure the proximal end portion of the catheter adapter, and a probe access portion. The probe access portion includes a needle free connector configured to receive a distal connector portion of the blood draw or probe delivery device, and the probe access portion is positioned in-line with the catheter adapter to provide a direct fluid path for a tube or probe of the blood draw or probe delivery device to be advanced through the probe access portion, the catheter adapter, and the catheter.

### CROSS-REFERENCE TO RELATED APPLICATION

The present application claims priority to Indian Provisional Application No. 202211028612 entitled “Vascular Access System with Integrated Catheter, Extension Set Providing Integrated Catheter Septum Access, Stabilization Features and Direct Probe Access” filed May 18, 2022, the entire disclosure of which is hereby incorporated by reference in its entirety.

### BACKGROUND OF THE INVENTION

### Field of the Invention

The present disclosure generally relates to vascular access systems such as, e.g., peripheral intravenous catheters (PIVCs). More particularly, the present disclosure relates to vascular access systems optimized for compatibility with blood draw and/or probe delivery devices.

### Description of Related Art

A catheter is commonly used to infuse fluids into vasculature of a patient. For example, the catheter may be used for infusing normal saline solution, various medicaments, or total parenteral nutrition. Furthermore, the catheter may also be used for withdrawing blood from the patient.

The catheter may be an over-the-needle peripheral intravenous catheter (PIVC). In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient. After proper placement of the needle, the clinician may temporarily occlude flow in the vasculature and remove the needle, leaving the catheter in place (i.e., “indwelled”) for future blood withdrawal and/or fluid infusion.

In order to complete blood draws from PIVCs having indwelled catheters, blood draw devices have been developed that are configured to overcome previous challenges related to blood draw through PIVCs such as, e.g., the possibility of catheter collapse, reduced blood flow due to debris built up on or within the catheter, etc. One such device, PIVO™ from Velano Vascular, Inc., is configured as a single-use device which temporarily attaches to a PIVC to draw a blood sample. Using an existing peripheral intravenous line as a conduit to the vasculature, the PIVO™ device advances a probe in the form of a flexible, internal flow tube through the PIVC, beyond the catheter tip, and into the vein to collect a blood sample. This flow tube is designed to extend beyond the suboptimal draw conditions around the indwelling line to reach vein locations where blood flow is optimal for aspiration. Once blood collection is complete, the flow tube is retracted, and the device is removed from the PIVC and discarded.

In addition to blood draw devices such as the PIVO™ device described above, similar devices for advancing a probe (e.g., a nickel titanium wire, guidewire, instrument, obturator, rod, wire with fluid path, and/or sensor) through an indwelling catheter and into a patient's vasculature have also been developed. However, existing vascular access devices are not generally optimized for compatibility with these blood draw and/or probe delivery devices, resulting in increased manipulation of the catheter access site when using the probe and, thus, increased risk of catheter dislodgement due to coupling, decoupling, and/or use of the blood draw or probe delivery device(s).

### SUMMARY OF THE INVENTION

In accordance with an aspect of the present disclosure, a vascular access system for use with a blood draw or probe delivery device is disclosed. The system includes a catheter adapter having a proximal end portion and a distal end portion, wherein a catheter extends from the distal end portion of the catheter adapter, and an extension set. The extension set includes a stabilization platform, a catheter adapter interface, wherein the catheter adapter interface is configured to secure the proximal end portion of the catheter adapter, and a probe access portion, wherein the probe access portion includes a needle free connector configured to receive a distal connector portion of the blood draw or probe delivery device, and wherein the probe access portion is positioned in-line with the catheter adapter to provide a direct fluid path for a tube or probe of the blood draw or probe delivery device to be advanced through the probe access portion, the catheter adapter, and the catheter.

In some embodiments, the catheter adapter interface extends from a top surface of the stabilization platform.

In some embodiments, the probe access portion is coupled to a top surface of the stabilization platform.

In some embodiments, the extension set further includes a septum access cannula extending distally from the catheter adapter interface.

In some embodiments, the septum access cannula includes a blunt cannula.

In some embodiments, the catheter adapter further includes an integrated catheter septum disposed within the proximal end portion thereof, and the integrated catheter septum is configured to receive the septum access cannula therein.

In some embodiments, the stabilization platform is angled such that the septum access cannula is angled toward a patient's skin surface.

In some embodiments, the catheter adapter interface is configured to non-removably secure the proximal end portion of the catheter adapter.

In some embodiments, the catheter adapter interface includes a plurality of snap features to secure the proximal end portion of the catheter adapter.

In some embodiments, the extension set further includes an intermediate tubing disposed between the proximal end portion of the catheter adapter and the probe access portion.

In some embodiments, the extension set further includes a proximal extension tubing extending from a side surface of the probe access portion distal to the needle free connector.

In some embodiments, the proximal extension tubing extends from the side surface of the probe access portion at an angle between 30°-150° relative to a longitudinal axis of the probe access portion.

In some embodiments, the probe access portion includes at least one of an offset side port from which the proximal extension tubing extends and a vortex feature within a fluid path of the probe access portion to direct fluid directed through the proximal extension tubing toward the needle free connector.

According to another aspect of the present disclosure, an extension set for use with a vascular access device is disclosed. The extension set includes a stabilization platform, a catheter adapter interface, wherein the catheter adapter interface is configured to secure a proximal end portion of a catheter adapter of the vascular access device, a septum access cannula extending distally from the catheter adapter interface, and a probe access portion, wherein the probe access portion includes a needle free connector configured to receive a distal connector portion of a blood draw or probe delivery device, and wherein the probe access portion is positioned in-line with the septum access cannula.

In some embodiments, the catheter adapter interface extends from a top surface of the stabilization platform.

In some embodiments, the probe access portion is coupled to a top surface of the stabilization platform.

In some embodiments, the stabilization platform is angled such that the septum access cannula is angled toward a patient's skin surface.

In some embodiments, the extension set further includes an intermediate tubing disposed between the proximal end portion of the catheter adapter and the probe access portion.

In some embodiments, the extension set further includes proximal extension tubing extending from a side surface of the probe access portion distal to the needle free connector.

In some embodiments, the probe access portion includes at least one of an offset side port from which the proximal extension tubing extends and a vortex feature within a fluid path of the probe access portion to direct fluid directed through the proximal extension tubing toward the needle free connector.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a perspective view of a vascular access system in accordance with an aspect of the present disclosure;

FIG. 2 is a perspective view of the vascular access system of FIG. 1 coupled to a blood draw device in accordance with an aspect of the present disclosure;

FIG. 3 is a perspective view of an extension set configuration for use with the vascular access system of FIG. 1 in accordance with an aspect of the present disclosure;

FIG. 4 is a perspective view of an extension set configuration for use with a vascular access system in accordance with another aspect of the present disclosure;

FIG. 5 is a perspective view of an extension set configuration for use with a vascular access system in accordance with another aspect of the present disclosure;

FIG. 6A is a top view of an extension set configuration for use with a vascular access system in accordance with another aspect of the present disclosure;

FIG. 6B is a side view of the extension set configuration of FIG. 6A;

FIG. 7A is a front view of a probe access portion in accordance with another aspect of the present disclosure;

FIG. 7B is a cross-sectional view of the probe access portion of FIG. 7A; and

FIG. 7C is a side view of the probe access portion of FIG. 7A;

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw or probe delivery device in preparation for or during use. Similarly, in this application, the terms “in the distal direction” and “distally” mean in the direction toward the connector portion of the fluid transfer device, and the terms “in the proximal direction” and “proximally” mean in the direction opposite the direction of the connector.

Embodiments of the present disclosure will primarily be described in the context of vascular access systems including an integrated peripheral IV catheter (PIVC). However, embodiments of the present disclosure equally extend to use with other catheter devices.

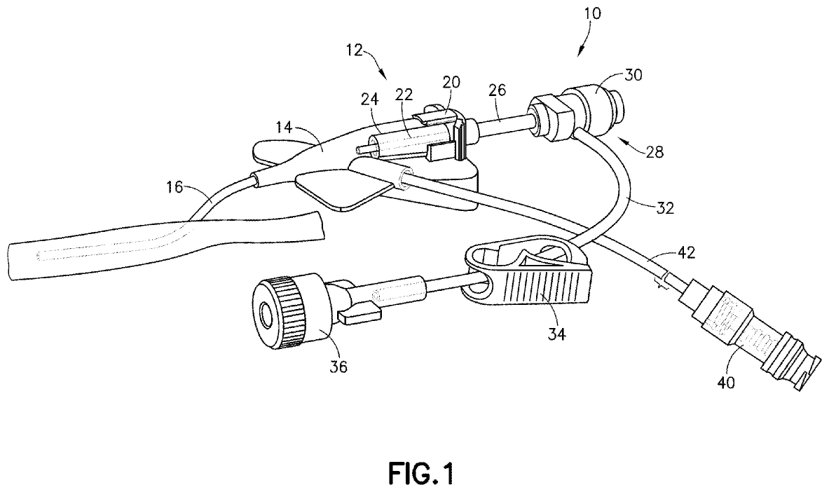

Referring to FIG. 1, a vascular access system 10 in accordance with an aspect of the present disclosure is illustrated. The vascular access system 10 includes an extension set 12 coupled to an integrated catheter adapter 14, with the catheter adapter 14 having a catheter 16 extending distally therefrom and configured for insertion into a patient's vasculature. The extension set 12 includes a stabilization platform 18 and a catheter adapter interface 20 extending vertically above an upper surface of the stabilization platform 18. A septum access cannula 22 extends distally from the catheter adapter interface 20, with the septum access cannula 22 configured to penetrate an integrated catheter septum 24 of the catheter adapter 14 to provide fluid communication between the catheter 16 and the septum access cannula 22. In some embodiments, the septum access cannula 22 is a blunt metal or plastic cannula, which may be formed separately from or integrated with the catheter adapter interface 20. Additionally, in some embodiments, the septum access cannula 22 may have a side hole (not shown) positioned distal to the catheter septum's distal surface for improved flushing of the catheter adapter 14. In other embodiments, the septum access cannula 22 may be a sharp cannula, and/or the septum access cannula may be initially covered with a cannula cover prior to connection to the catheter adapter 14.

Vascular access system 10 further includes a probe access portion 28, with probe access portion 28 including a needle free connector 30. In some embodiments, the needle free connector 30 is configured as a split septum-type needle free connector. However, in other embodiments, any appropriate connector may be used, including other forms of needle free connectors (integrated or removable), needle connectors (e.g., PRN connectors), etc. The needle free connector 30 is coupled to the catheter adapter interface 20 via intermediate tubing 26 such that the needle free connector 30 is in fluid communication with, and is generally aligned with, the septum access cannula 22.

The probe access portion 28 further includes proximal extension tubing 32 extending from a side portion of the needle free connector 30, wherein the proximal extension tubing 32 is in fluid communication with the needle free connector 30 and, thus, is also in fluid communication with the intermediate tubing 26 and septum access cannula 22. A proximal luer adapter 36 may be coupled to the proximal end of proximal extension tubing 32, and a clamping device 34 may be placed in-line over the proximal extension tubing 32 so as to enable selective occlusion of the proximal extension tubing 32. While the proximal luer adapter 36 shown in FIG. 1 is only a single port adapter, it is to be understood that proximal luer adapter 36 may be include more than one port. The proximal luer adapter 36 is configured to receive various devices for, e.g., fluid administration and flushing of the vascular access system 10.

In some embodiments, the catheter adapter 14 includes a side branch 38, with a tube set 42 extending from the side branch 38 and a connector portion 40 at the proximal end portion of the tube set 42. The connector portion 40 may be any appropriate connector configuration capable of attachment to a fluid administration or flushing device. However, in some embodiments, side branch 38 and tube set 42 may be omitted, with catheter adapter 14 only fluidly coupled to the probe access portion 28.

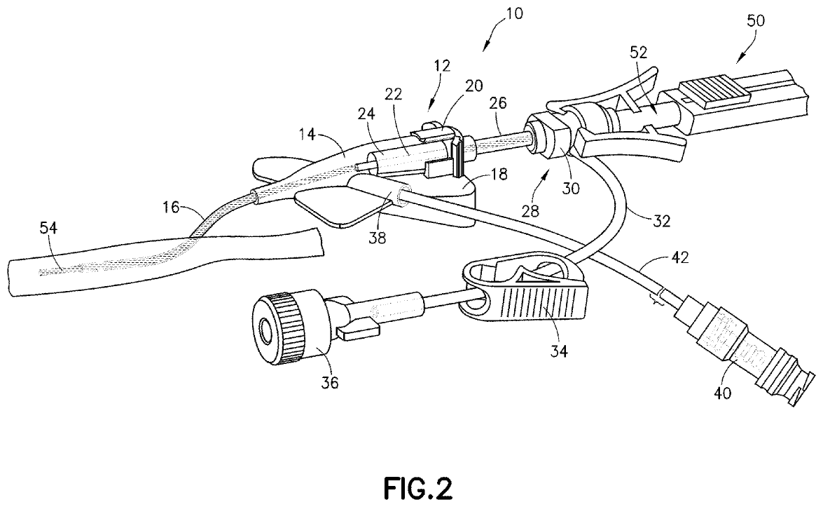

Referring now to FIG. 2, vascular access system 10 is shown with a blood draw device 50 coupled thereto. More specifically, a distal connector portion 52 of blood draw device 50 is configured to releasably couple the blood draw device 50 to the needle free connector 30 of the probe access portion 28. In the embodiment shown in FIG. 2, the blood draw device 50 includes a flexible tube 54 which may be selectively advanced through the intermediate tubing 26, the septum access cannula 22, the catheter adapter 14, and beyond the distal tip of the catheter 16 such that a blood sample may be drawn by the blood draw device 50 into an appropriate sample collection container (not shown). However, it is to be understood that the vascular access system 10 is not limited to use with blood draw device 50, but can instead be used with any device capable of advancing an elongated tube or probe (e.g., a nickel titanium wire, guidewire, instrument, obturator, rod, wire with fluid path and/or sensor, etc.).

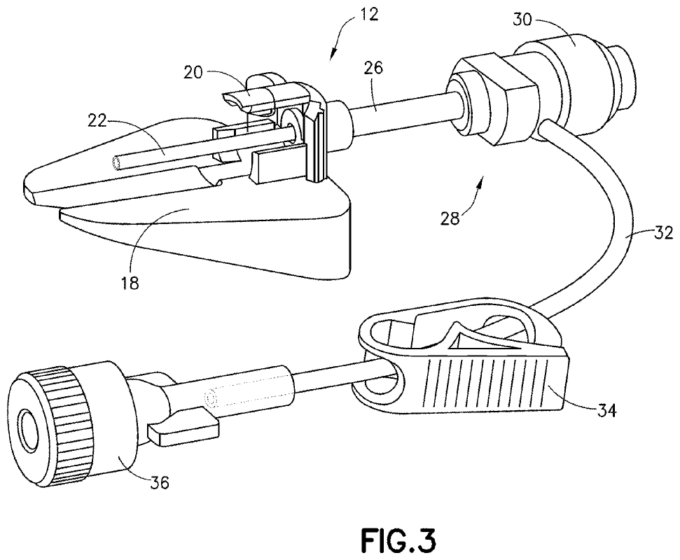

Referring to FIG. 3, the extension set 12 of vascular access system 10 is shown in greater detail. In particular, the stabilization platform 18 is angled such that the septum access cannula 22 is directed at an optimized angle relative to the patient's skin. In this way, when the catheter adapter 14 is coupled to the catheter adapter interface 20, the catheter 16 enters the patient's vasculature at an optimized angle. Additionally, the angle of stabilization platform 18 also provides an optimal angle for advancement of the tube or probe (e.g., tube 54 shown in FIG. 2) through the vascular access system 10. In some embodiments, the stabilization platform 18 is integrally formed with the catheter adapter interface 20 (e.g., an over-molded elastomeric material). In other embodiments, the stabilization platform 18 may be formed separately from the catheter adapter interface 20. In some embodiments, the stabilization platform 18 may include an adhesive incorporated thereon to improve securement to the patient's skin. Alternatively, in other embodiments, the stabilization platform 18 may rely on only a top dressing for securement to the patient's skin. Additionally and/or alternatively, other aspects of the extension set 12 may include stabilization features such as, e.g., wings. Such stabilization features may be provided on, e.g., the catheter adapter 14, the catheter adapter interface 20, the needle free connector 30, etc. Or, in other embodiments, the extension set 12 may have no stabilization feature(s).

With the features of the vascular access system 10 shown and described above with respect to FIGS. 1-3, direct and substantially axial access by a probe device (e.g., blood draw device 50) to the catheter 16 may be provided via the intermediate tubing 26 and the probe access portion 28, which minimizes the overall height of the system and provides flexibility in the system, thereby reducing manipulation at the catheter insertion site. Additionally, because the stabilization platform 18 is located beneath the catheter adapter 14, risk of access site manipulation and/or catheter movement or dislodgement is also reduced. Furthermore, vascular access system 10 enables easier securement of the extension set 12 with a dressing, while providing a proximal slot in the dressing for the probe access portion 28 and proximal extension tubing 32 to exit the dressing for access via a probe device, fluid infusion device, etc., with minimal disturbance to the catheter access site.

In some embodiments, the catheter adapter interface 20 is configured to non-removably secure the catheter adapter 14 upon placement of the integrated catheter septum 24 over the septum access cannula 22. Alternatively, in other embodiments, the catheter adapter interface 20 and/or catheter adapter 14 may be configured such that the catheter adapter 14 is removably secured to the catheter adapter interface 20.

Additionally, in some embodiments, each internal junction of the respective components of vascular access system 10 may have the same (or an increasing) internal diameter in the distal direction (i.e., in the direction from the needle free connector 30 to the catheter 16), which allows the tube or probe advanced by, e.g., the blood draw device 50 to avoid any steps or ridges which may catch or otherwise slow the tube or probe as it moves distally through the system.

Furthermore, in some embodiments, the probe access portion 28 may include an in-line anti-reflux valve (not shown) located distally to the needle free connector 30, with the anti-reflux valve being a separate component from the needle free connector 30 or integrally formed with the needle free connector 30. Additionally and/or alternatively, the needle free connector 30 may include features to improve flushing of the system, as will be described in further detail below.

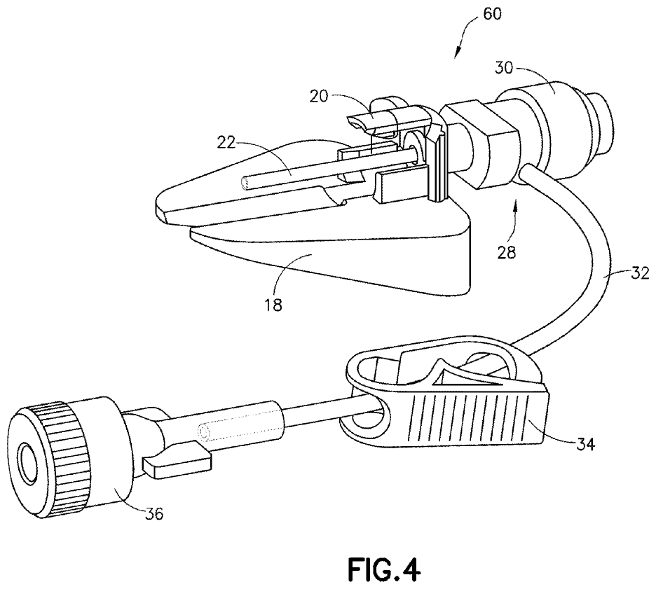

Next, referring to FIG. 4, an extension set 60 in accordance with another aspect of the present disclosure is shown. Extension set 60 is substantially similar to extension set 12 described above with respect to FIGS. 1-3, and thus like reference numerals are used throughout. However, unlike extension set 12, extension set 60 does not include a length of intermediate tubing between the probe access portion 28 and the catheter adapter interface 20. As the probe access portion 28 (and, thus, the needle free connector 30) is directly coupled to the catheter adapter interface 20, extension set 60 provides a shorter path through the system in which the probe or tube of, e.g., a blood draw device must travel. In such a configuration, the stabilization platform 18 can be platform may be coupled to either the catheter adapter interface 20 (as shown in FIG. 4), or it could be coupled to and extend beneath the probe access portion 28.

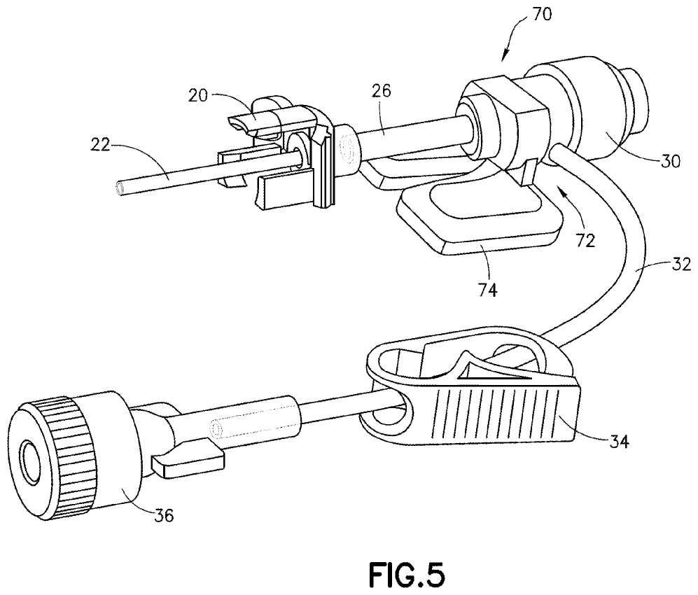

Referring now to FIG. 5, an extension set 70 in accordance with another aspect of the present disclosure is shown. Unlike extension sets 10, 60 described above, extension set 70 includes a probe access portion 72 that is coupled to a stabilization platform 74 such that the stabilization platform 74 extends below the probe access portion 72 (and, thus, the needle free connector 30). The intermediate tubing 26 extends distally from the needle free connector 30, and the catheter adapter interface 20 and septum access cannula 22 extend distally from the intermediate tubing 26. Accordingly, the catheter adapter interface 20 is able to move relative to the probe access portion 72, but the stabilization platform 74 still provides for a stabilized extension set to reduce manipulation of the catheter access site when a device such as, e.g., blood draw device 50 shown in FIG. 2 is coupled to the needle free connector 30.

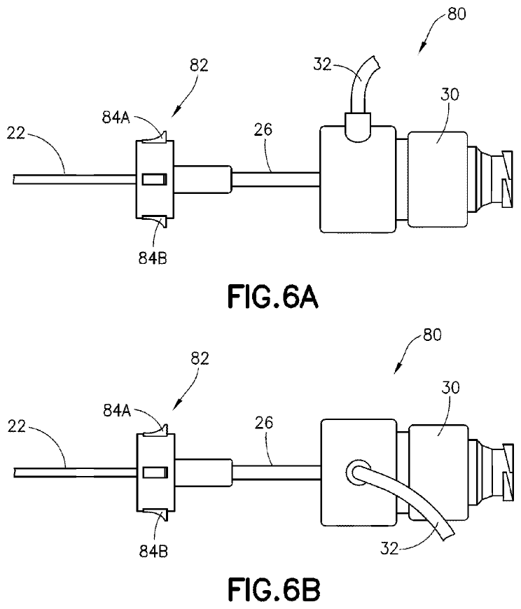

Next, referring to FIGS. 6A and 6B, an extension set 80 in accordance with another aspect of the present disclosure is shown. As noted above, in some embodiments, the catheter adapter interface may be configured to non-removably or removably secure the catheter adapter upon placement of the integrated catheter septum 24 over the septum access cannula 22. To secure the catheter adapter, extension set 80 includes a catheter adapter interface 82 having a plurality of snap features 84A, 84B, with the snap features 84A, 84B providing for secure and non-removable coupling of the catheter adapter (not shown) to the catheter adapter interface 82. In some embodiments, the snap features 84A, 84B are internal structures capable of holding a proximal end portion of the catheter adapter. Additionally and/or alternatively, other securement features such as, e.g., a threaded connection or a slip luer connection may be used to secure the proximal end portion of the catheter adapter to the catheter adapter interface.

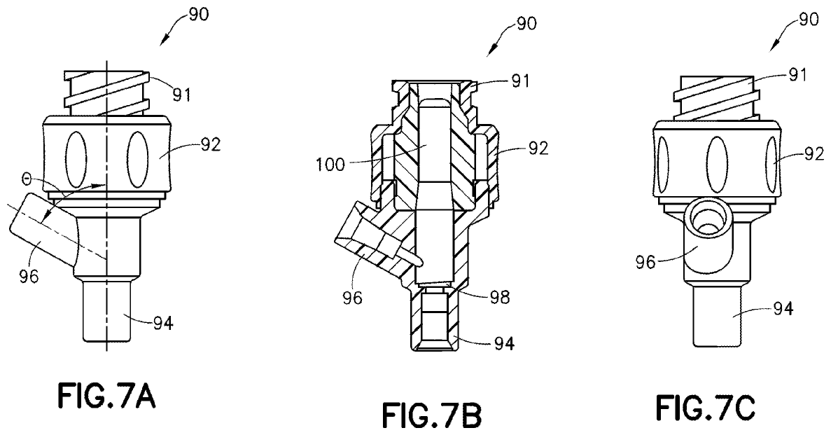

Referring now to FIGS. 7A-7C, a probe access portion 90 in accordance with another aspect of the present disclosure is shown. The probe access portion 90 is usable with the various extension sets described above with respect to FIGS. 1-6B, and may include features to improve flushing of the probe access portion 90 and portions of the extension set coupled thereto. The probe access portion 90 includes a needle free connector 92 having a proximal end portion 91 configured for coupling to, e.g., a probe device such as a blood draw device. The needle free connector 92 defines a flow path 100 extending between the proximal end portion 91 and a distal end portion 94, with the distal end portion 94 couplable to, e.g., intermediate tubing.

The probe access portion 90 also includes a side port 96, wherein the side port may be coupled to, e.g., proximal extension tubing for fluid injection, flushing, etc. In some embodiments, the side port 96 may be offset from a center of the flow path 100, as is shown in FIG. 7C. The offset of the side port 96 is configured to cause fluid entering the probe access portion 90 via the side port 96 to enter along an interior surface of the flow path 100 and cause a vortex or otherwise redirect fluid proximally within the probe access portion 90 to aid flushing of the needle free connector 92. In some embodiments, the probe access portion 90 further includes an internal structure 98 configured to create a vortex or otherwise redirect fluid when fluid enters the needle free connector probe access portion 90 via the side port 96. In some embodiments, the offset and vortex-creating feature is the same or similar to the flushing features shown and described in U.S. Patent Application Publication No. 2021/0220548, which is hereby incorporated by reference in its entirety.

The body of the probe access portion 90 defines a longitudinal axis extending between the proximal end portion 91 and the distal end portion 94, with the side port 96 extending from the body at an angle θ of e.g., 30°-150° relative to the longitudinal axis of the body. In one embodiment, the side port 96 extends from the body at an angle of 60° relative to the longitudinal axis of the body. The side port 96 extends at the angle θ toward the distal end portion 94, although other suitable arrangements may be utilized. Furthermore, in some embodiments, the probe access portion 90 is formed as a single part. However, in other embodiments, the probe access portion may be formed of more than one part.

While several embodiments of vascular access systems and/or extension sets were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A vascular access system for use with a blood draw or probe delivery device, the system comprising:
a catheter adapter having a proximal end portion and a distal end portion, wherein a catheter extends from the distal end portion of the catheter adapter; and
an extension set, the extension set comprising:
a stabilization platform,
a catheter adapter interface, wherein the catheter adapter interface is configured to secure the proximal end portion of the catheter adapter, and
a probe access portion, wherein the probe access portion comprises a needle free connector configured to receive a distal connector portion of the blood draw or probe delivery device, and wherein the probe access portion is positioned in-line with the catheter adapter to provide a direct fluid path for a tube or probe of the blood draw or probe delivery device to be advanced through the probe access portion, the catheter adapter, and the catheter.

2. The system of claim 1, wherein the catheter adapter interface extends from a top surface of the stabilization platform.

3. The system of claim 1, wherein the probe access portion is coupled to a top surface of the stabilization platform.

4. The system of claim 1, wherein the extension set further comprises a septum access cannula extending distally from the catheter adapter interface.

5. The system of claim 4, wherein the septum access cannula comprises a blunt cannula.

6. The system of claim 4, wherein the catheter adapter further comprises an integrated catheter septum disposed within the proximal end portion thereof, and wherein the integrated catheter septum is configured to receive the septum access cannula therein.

7. The system of claim 4, wherein the stabilization platform is angled such that the septum access cannula is angled toward a patient's skin surface.

8. The system of claim 1, wherein the catheter adapter interface is configured to non-removably secure the proximal end portion of the catheter adapter.

9. The system of claim 8, wherein the catheter adapter interface comprises a plurality of snap features to secure the proximal end portion of the catheter adapter.

10. The system of claim 1, wherein the extension set further comprises an intermediate tubing disposed between the proximal end portion of the catheter adapter and the probe access portion.

11. The system of claim 1, wherein the extension set further comprises a proximal extension tubing extending from a side surface of the probe access portion distal to the needle free connector.

12. The system of claim 11, wherein the proximal extension tubing extends from the side surface of the probe access portion at an angle between 30°-150° relative to a longitudinal axis of the probe access portion.

13. The system of claim 11, wherein the probe access portion includes at least one of an offset side port from which the proximal extension tubing extends and a vortex feature within a fluid path of the probe access portion to direct fluid directed through the proximal extension tubing toward the needle free connector.

14. An extension set for use with a vascular access device, the extension set comprising:
a stabilization platform;
a catheter adapter interface, wherein the catheter adapter interface is configured to secure a proximal end portion of a catheter adapter of the vascular access device;
a septum access cannula extending distally from the catheter adapter interface; and
a probe access portion, wherein the probe access portion comprises a needle free connector configured to receive a distal connector portion of a blood draw or probe delivery device, and wherein the probe access portion is positioned in-line with the septum access cannula.

15. The extension set of claim 14, wherein the catheter adapter interface extends from a top surface of the stabilization platform.

16. The extension set of claim 14, wherein the probe access portion is coupled to a top surface of the stabilization platform.

17. The extension set of claim 14, wherein the stabilization platform is angled such that the septum access cannula is angled toward a patient's skin surface.

18. The extension set of claim 14, further comprising an intermediate tubing disposed between the proximal end portion of the catheter adapter and the probe access portion.

19. The extension set of claim 14, further comprising proximal extension tubing extending from a side surface of the probe access portion distal to the needle free connector.

20. The extension set of claim 19, wherein the probe access portion includes at least one of an offset side port from which the proximal extension tubing extends and a vortex feature within a fluid path of the probe access portion to direct fluid directed through the proximal extension tubing toward the needle free connector.
